(19) United States
(12) Patent Application Publication
(10) Pub. No.: US 2008/0318986 A1
(43) Pub. Date: Dec. 25, 2008

(54) Ddc Compositions

(76) Inventors: Rhett M. Schiffman, Laguna Beach, CA (US); Patrick M. Hughes, Aliso Viejo, CA (US); Jerald Gordon, Pittsburgh, PA (US); Eric Romanowski, Pittsburgh, PA (US)

Correspondence Address: ALLERGAN, INC., 2525 DUPONT DRIVE, T2-7H, IRVINE, CA, 92612-1599, US

(21) Appl. No.: 11/997,397

(22) PCT Filed: Oct. 11, 2006
(86) PCT No.: PCT/US2006/039986

§ 371 Date: Aug. 12, 2008

Related U.S. Application Data
(60) Provisional application No. 60/596,696, filed on Oct. 13, 2005.

(51) Int. Cl. A61K 31/513; A61P 27/02; A61P 31/12

(52) U.S. Cl. 514/274

(57) ABSTRACT

A composition comprising from 1% to 3% dDC which is aqueous and ophthalmically acceptable is disclosed herein.

### CROSS REFERENCE TO RELATED APPLICATIONS

This application is based on, and claims the benefit of, U.S. Provisional Application No. 60/596,696, filed Oct. 13, 2005, and which is incorporated herein by reference.

### DESCRIPTION

Viral conjunctivitis, known commonly as pink eye, is a common highly contagious disease. 2′,3′-Dideoxycytidine (dDC), also known as zalcitabine, is an antiviral drug which is used to treatment of HIV patients having the structure shown below.

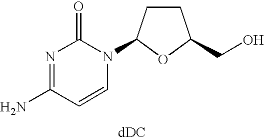

Kaneko et. al. (J. Jpn. Ophthalm. Soc. 104: 786-791 (2000)) stated “[f]uture research on development of HPMPC and dDC as eye drops for AdV condunctivitis are promising.” In a later related study, Kaneko et. al. (J. Jpn. Opthalmol. Soc. 107:196-201 (2003)) found that NMSO₃, HPMPC, and dDC inhibited adenoviral strains, but that NMSO₃ had the lowest EC50 values. Keneko further pointed out that there may be problems with adverse reactions related to the cytotocity of nucleic acid derivatives such as HPMPC and dDC.

WO2004/087203 discloses “methods and composition of an immunostimulatory nucleic acid in oil-in-water emulsions for topical delivery.” It further mentions that “[t[he immunostimulatory nucleic acid may be administered to the skin or to the mucosa. Mucosal administration include oral, ocular, nasal, vaginal, rectal and the like” and that “[i]n some embodiments the anti-viral agent is selected from the group consisting of Acemannan; Acyclovir; Acyclovir Sodium; Adefovir; Alovudine; Alvircept Sudotox; Amantadine Hydrochloride; Aranotin; Arildone; Atevirdine Mesylate; Avridine; Cidofovir Cipamfylline; Cytarabine Hydrochloride; Delavirdine Mesylate; Desciclovir Didanosine; Disoxaril; Edoxudine; Enviradene; Enviroxime; Famciclovir; Famotine Hydrochloride; Fiacitabine; Fialuridine; Fosarilate; Foscamet Sodium; Fosfonet Sodium; Ganciclovir; Ganciclovir Sodium; Idoxuridine; Kethoxal; Lamivudine; Lobucavir; Memotine Hydrochloride; Methisazone; Nevirapine Penciclovir; Pirodavir; Ribavirin; Rimantadine Hydrochloride; Saquinavir Mesylate; Somantadine Hydrochloride; Sorivudine; Statolon; Stavudine; Tilorone Hydrochloride; Trifluridine; Valacyclovir Hydrochloride; Vidarabine; Vidarabine Phosphate; Vidarabine Sodium Phosphate; Viroxime; Zalcitabine; Zidovudine; and Zinviroxime.”

A composition comprising from 1% to 3% dDC which is aqueous and ophthalmically acceptable is disclosed herein.

Unless otherwise indicated, all concentrations given as % in the specification and the claims herein are intended to mean % (weight/volume).

Another composition comprises from 1% to 2% dDC.

Another composition comprises 1.65% dDC.

A method comprising administering an eye drop to a person for the treatment of a viral infection, wherein said eye drop comprises from 300 μg to 900 μg dDC is disclosed herein. In other words, the eye receives no more than 300 μL in each individual treatment. This method may be practiced on both eyes simultaneously. In other words, a person may receive a treatment or dose of 300 μg to 900 μg dDC to one eye, and another treatment or dose of 300 μg to 900 μg dDC to the second eye, at or around the same time.

In one embodiment, a dose of dDC having from 300 μg to 900 μg dDC, is administered no more than 6 times a day. These six doses may occur in any combination to one or both eyes. In other words, in one embodiment a dose having 300 μg to 900 μg dDC is administered to both eyes of the person no more than 3 times a day. In another embodiment, a dose having 300 μg to 900 μg dDC is administered to only one eye no more than 6 times a day. In other embodiments, both eyes receive 1 or more dose of 300 μg to 900 μg dDC per day, but the total number of doses does not exceed six. For example, one eye may receive 2 doses and the other eye receive 4 doses; one eye may receive 1 dose and the other eye may receive 5 doses; one eye may receive 2 doses and the other eye may receive 3 doses; etc.

An eye drop comprising from 300 μg to 900 μg dDC is disclosed herein.

In another embodiment the eye drop comprises from 300 μg to 600 μg dDC.

The compounds, methods, medicaments, and compositions disclosed herein are useful for the treatment of viral infections affecting the ocular surface or other parts of the eye.

Also contemplated herein is a kit comprising a dispenser and a label, said dispenser containing a composition disclosed herein, and said dispenser being capable of providing drops of said composition suitable for topical administration to an eye. Said kit may also contain a label indicating administration of said drops of said composition to an eye of a mammal.

Diseases or conditions which may be treated include viral conjunctivitis, viral keratitis, viral trabeculitis, viral iritis, viral scleritis, viral blethritis, viral vitritis, and other viral uveitides.

While not intending to limit the scope of the invention in anyway, examples of viruses affecting the eye include

adenovirus (all serotypes);

Herpes virus including, but not limited to simplex type 1 & 2, varicella zoster, Epstein Barr, cytomegalovirus, and human herpes virus 6, 7, and 8, and the like;

picornaviruses including, but not limited to enterovirus, Coxsackie, and the like;

poxvirus molluscum contagiosum, vaccinia).

paramyxovirus including, but not limited to measles, mumps, Newcastile, and the like;

West Nile Virus; and

rubella virus and the like.

In one embodiment, the compositions are used to treat viral conjunctivitis or viral keratitis.

Reference to dDC herein should be broadly interpreted to mean dDC or a pharmaceutically acceptable salt thereof. A pharmaceutically acceptable salt is any salt that retains the activity of the parent compound and does not impart any additional deleterious or untoward effects on the subject to which it is administered and in the context in which it is administered compared to the parent compound. A pharmaceutically acceptable salt also refers to any salt which may form in vivo as a result of administration of an acid, another salt, or a prodrug which is converted into an acid or salt.

Pharmaceutically acceptable salts of acidic functional groups may be derived from organic or inorganic bases. The salt may comprise a mono or polyvalent ion. Of particular interest are the inorganic ions, lithium, sodium, potassium, calcium, and magnesium. Organic salts may be made with amines, particularly ammonium salts such as mono-, di- and trialkyl amines or ethanol amines. Salts may also be formed with caffeine, tromethamine and similar molecules. Hydrochloric acid or some other pharmaceutically acceptable acid may form a salt with a compound that includes a basic group, such as an amine or a pyridine ring.

In the case that a concentration of dDC is disclosed, it should be taken to be the concentration of dDC in neutral form whether or not the dDC is present as a salt.

A “prodrug” is a compound which is converted to a therapeutically active compound after administration, and the term should be interpreted as broadly herein as is generally understood in the art. While not intending to limit the scope of the invention, conversion may occur by hydrolysis of an ester group or some other biologically labile group. Generally, but not necessarily, a prodrug is inactive or less active than the therapeutically active compound to which it is converted. A metabolite is broadly defined as a compound which is formed in vivo from the disclosed compound.

The amount of the presently useful compound or compounds administered is, of course, dependent on the therapeutic effect or effects desired, on the specific mammal being treated, on the severity and nature of the mammal's condition, on the manner of administration, on the potency and pharmacodynamics of the particular compound or compounds employed, and on the judgment of the prescribing physician.

A liquid which is ophthalmically acceptable is formulated such that it can be administered topically to the eye. The comfort should be maximized as much as possible, although sometimes formulation considerations (e.g. drug stability) may necessitate less than optimal comfort. In the case that comfort cannot be maximized, the liquid should be formulated such that the liquid is tolerable to the patient for topical ophthalmic use. Additionally, an ophthalmically acceptable liquid should either be packaged for single use, or contain a preservative to prevent contamination over multiple uses.

For ophthalmic application, solutions or medicaments are often prepared using a physiological saline solution as a major vehicle. Ophthalmic solutions should preferably be maintained at a comfortable pH with an appropriate buffer system. The formulations may also contain conventional, pharmaceutically acceptable preservatives, stabilizers and surfactants.

Preservatives that may be used in the pharmaceutical compositions of the present invention include, but are not limited to, benzalkonium chloride, chlorobutanol, thimerosal, phenylmercuric acetate and phenylmercuric nitrate. A useful surfactant is, for example, Tween 80. Likewise, various useful vehicles may be used in the ophthalmic preparations of the present invention. These vehicles include, but are not limited to, polyvinyl alcohol, povidone, hydroxypropyl methyl cellulose, poloxamers, carboxymethyl cellulose, hydroxyethyl cellulose and purified water.

Tonicity adjustors may be added as needed or convenient. They include, but are not limited to, salts, particularly sodium chloride, potassium chloride, mannitol and glycerin, or any other suitable ophthalmically acceptable tonicity adjustor.

Various buffers and means for adjusting pH may be used so long as the resulting preparation is ophthalmically acceptable. Accordingly, buffers include acetate buffers, citrate buffers, phosphate buffers and borate buffers. Acids or bases may be used to adjust the pH of these formulations as needed.

In a similar vein, an ophthalmically acceptable antioxidant for use in the present invention includes, but is not limited to, sodium metabisulfite, sodium thiosulfate, acetylcysteine, butylated hydroxyanisole and butylated hydroxytoluene.

Other excipient components which may be included in the ophthalmic preparations are chelating agents. A useful chelating agent is edetate disodium, although other chelating agents may also be used in place or in conjunction with it.

The ingredients are usually used in the following amounts:

| Ingredient | Amount (% w/v) |
|---|---|
| preservative | 0-0.10 |
| vehicle | 0-40 |
| tonicity adjustor | 1-10 |
| buffer | 0.01-10 |
| pH adjustor | q.s. pH 4.5-7.5 |
| antioxidant | as needed |
| surfactant | as needed |
| purified water | as needed to make 100% |

The actual dose of the active compounds of the present invention depends on the specific compound, and on the condition to be treated; the selection of the appropriate dose is well within the knowledge of the skilled artisan.

The foregoing description details specific methods and compositions that can be employed to practice the present invention, and represents the best mode contemplated. However, it is apparent for one of ordinary skill in the art that further compounds with the desired pharmacological properties can be prepared in an analogous manner, and that the disclosed compounds can also be obtained from different starting compounds via different chemical reactions. Similarly, different pharmaceutical compositions may be prepared and used with substantially the same result. Thus, however detailed the foregoing may appear in text, it should not be construed as limiting the overall scope hereof; rather, the ambit of the present invention is to be governed only by the lawful construction of the appended claims.

### EXAMPLE 1

TABLE 1

| Ingredient | amount |
|---|---|
| dideoxycytidine (ddC) | 1.5% |
| Methocel A4M Premium | 0.5% |
| Carbapol 934P | 0.3% |
| Glycerin | 2.2% |
| pH | 4.0 |

The formulation shown in Table 1 is manufactured in one part. The Carbapol 934P was added to a 0.5% methocel solution. Glycerin is then added with mixing followed by dDC. The pH is then adjusted to 4.0 with 1 N sodium hydroxide or 1 N hydrochloric acid.

One 35 μL drop of this composition is administered to each eye of a person suffering from viral conjunctivitis twice a day. Reduction in the infection begins within a few hours of the dose, and complete recovery occurs after a few days of treatment.

What is claimed is:

1. A composition comprising from 1% to 3% dDC which is aqueous and ophthalmically acceptable.

2. The composition of claim 1 comprising from 1% to 2% dDC.

3. The composition of claim 2 comprising 1.5% dDC.

4. The composition of claim 3 comprising 1.5% ddC, 0.5% Methocel A4M Premium, 0.3% Carbapol 934P, 2.2% Glycerin, and having a pH of 4.0.

5. An eye drop comprising from 300 μg to 900 μg dDC.

6. A method comprising administering an eye drop to a mammal for the treatment of a viral infection, wherein said eye drop comprises from 300 μg to 900 μg dDC.

7. The method of claim 6 wherein said viral infection is viral vitritis.

8. The method of claim 6 wherein said eye drop comprises from 300 μg to 600 μg dDC.
